(19) United States
(12) Patent Application Publication
(10) Pub. No.: US 2017/0266098 A1
(43) Pub. Date: Sep. 21, 2017

(54) AGENT AND METHOD FOR THE TEMPORARY SHAPING OF KERATIN-CONTAINING FIBERS

(71) Applicant: Henkel AG & Co. KGaA, Duesseldorf (DE)

(72) Inventors: Thorsten Knappe, Schenefeld (DE); Thomas Foerster, Duesseldorf (DE); Rolf Bayersdoerfer, Hamburg (DE)

(73) Assignee: Henkel AG & Co. KGaA, Duesseldorf (DE)

(21) Appl. No.: 15/532,145

(22) PCT Filed: Nov. 2, 2015
(86) PCT No.: PCT/EP2015/075380

§ 371 Date: Jun. 1, 2017

(30) Foreign Application Priority Data
Dec. 10, 2014 (DE) 10 2014 225 433.7

(51) Int. Cl. A61K 8/81; A61Q 5/06

(52) U.S. Cl. CPC A61K 8/8182; A61Q 5/06; A61K 2800/5422; A61K 2800/5426

(57) ABSTRACT

The disclosure relates to a cosmetic product, comprising: a) a cosmetic preparation, containing, in relation to its total weight, a1) from about 45 to about 99 wt.-% of at least one polar solvent; a2) from about 0.1 to about 6.0 wt.-% of a cationic polymer; and a3) from about 0.1 to about 12 wt.-% of a non-ionic polymer; b) a device for flash evaporation of the cosmetic preparation a), and to a method using corresponding products and the use of the cosmetic preparation a) as process material in a device for flash evaporation.

### CROSS-REFERENCE TO RELATED APPLICATION

This application is a U.S. National-Stage entry under 35 U.S.C. §371 based on International Application No. PCT/EP2015/075380, filed Nov. 2, 2015 which was published under PCT Article 21(2) and which claims priority to German Application No. 102014225433.7, filed Dec. 10, 2014, which are all hereby incorporated in their entirety by reference.

### TECHNICAL FIELD

The present patent application relates to the technical field of temporary shaping of keratin-containing fibers, in particular human hair. The subject matter of the patent application is specific hair cosmetic formulations which are suitable for application to keratin-containing fibers by employing a flash vaporization process. In addition the use of these hair cosmetic formulations in devices for flash vaporization and methods for temporary shaping of keratin-containing fibers are the subject matter of the present patent application.

### BACKGROUND

Today in general a hair style that looks attractive is considered to be an essential part of a well-groomed appearance because of current fashion trends, the chic hairstyles are often those that can be created and/or maintained for a longer period of time, even up to several days, for many types of hair only by using hair-setting ingredients. Therefore, hair treatment agents, which serve to impart a prominent or temporary shape to hair play an important role. In permanent shaping, the chemical structure of the keratin-containing fibers is modified by reduction and oxidation, but there are no such modifications to the chemical structure in temporary shaping. Corresponding means for temporary shaping usually contain synthetic polymers and/or waxes as the active hair styling ingredient.

The most important property of an agent for temporary shaping of keratin-containing fibers, hereinafter also referred to as styling agents, is to impart the strongest possible hold to the treated fibers in the newly modeled form, i.e., a form imposed on the fibers. If the keratinic fibers are human hair, then we also speak of a strong hair style hold or a high degree of hold of the styling agent. The hair style hold is determined essentially by the type and amount of hair setting agents used, but there may also be an influence of the additional ingredients of the styling agent as well as the form of application.

In the area of temporary shaping of keratinic fibers, spray application of corresponding cosmetic preparations has been found to be of a great importance, wherein the preparations are usually applied as pump sprays or aerosol sprays. To do so, the cosmetic preparations are fabricated in a dispensing device from which they are sprayed either by employing mechanical action of force or with the help of a propellant through a valve. Both of these methods have obvious disadvantages. Whereas pump sprays are usually unsuitable for a long-lasting, uniform spray application of cosmetic hair preparations, aerosol sprays are based on the use of propellants or propellant gases, which do not manifest any cosmetic effect, and on the other hand, which may be a source of risk to the consumer if handled improperly.

Against this background, there is a need for alternative methods for atomization of hair cosmetic preparations. Flash vaporization has proven to be one such alternative spraying method. In this method, which is described in the International Patent Application WO 2001/83071 A1 (Henkel), for example, a liquid or pasty solvent-based composition is heated in a sealed space to a temperature above the boiling point of the solvent, so that an excess pressure is created in the composition. On releasing (throttling) the pressure, the liquid evaporates and then can be atomized by employing a suitable nozzle, for example.

Even if flash vaporization is fundamentally successful for spray application of hair cosmetic preparations, at the same time, not all hair cosmetic preparations can be atomized by employing a flash vaporization process. On the one hand, this is due to the heating of the cosmetic preparation required for flash vaporization and, on the other hand, to the specifics of the spray mist formed by flash vaporization, for example, the resulting droplet size and droplet density in the spray mist.

### BRIEF SUMMARY

A cosmetic product is provided herein. The cosmetic product includes a cosmetic preparation including, based on its total weight, from about 45 to about 99 wt % of at least one polar solvent, from about 0.1 to about 6.0 wt % cationic polymer, and from about 0.1 to about 12 wt % nonionic polymer. The cosmetic product further incudes a device for flash vaporization of the cosmetic preparation.

A cosmetic preparation is also provided herein. The cosmetic preparation includes, based on its total weight, from about 45 to about 99 wt % of at least one polar solvent, from about 0.1 to about 6.0 wt % cationic polymer, and from about 0.1 to about 12 wt % nonionic polymer. The cosmetic preparation is utilized as a material in a device for flash vaporization.

A method for temporary shaping of keratin-containing fibers is also provided herein. The method including treating the keratin-containing fibers with a cosmetic preparation by employing a device for flash vaporization. The cosmetic preparation includes, based on its total weight, from about 45 to about 99 wt % of at least one polar solvent, from about 0.1 to about 6.0 wt % cationic polymer, and from about 0.1 to about 12 wt % nonionic polymer.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object of the present disclosure was therefore to make available specific hair cosmetic preparations for temporary shaping of keratin-containing fibers, said preparations being suitable for targeted spray application by employing a device for flash vaporization due to the chemical and physical properties. In addition, the preparations should be suitable for achieving a high degree of hold, in particular a high degree of long-term hold and a high volume effect after application by employing a flash vaporization method. It has been found that, from the variety of known polymer preparation that are effective as hair cosmetics, to achieve this object, solvent-based preparations containing a combination of cationic and nonionic polymers are suitable in particular.

A first subject matter of the present disclosure is thus a cosmetic product comprising:
a) a cosmetic preparation containing, based on its total weight
a1) from about 45 to about 99 wt % of at least one polar solvent;
a2) from about 0.1 to about 6.0 wt % cationic polymer;
a3) from about 0.1 to about 12 wt % nonionic polymer;
b) a device for flash vaporization of the cosmetic preparation a).

The cosmetic preparation a) is preferably liquid. The cosmetic preparation a) may be in the form of a solution or dispersion, for example an emulsion or suspension. Preferred cosmetic preparations a) are in the form of a solution or a suspension.

The cosmetic preparation as contemplated herein contains as the first essential ingredient from about 45 to about 99 wt % of at least one polar solvent a1). Preferred cosmetic products are characterized in that the amount by weight of the polar solvent a1) in the total weight of the cosmetic preparation a) is from about 70 to about 98 wt % preferably from about 80 to about 95 wt %. Corresponding agents are characterized by a good cosmetic effect with good applicability at the same time.

To improve the application properties of cosmetic preparations as contemplated herein while at the time minimizing the thermal load of any active ingredients or additives in the course of the flash vaporization process, it has proven to be advantageous to use polar solvents a1) which have a boiling point (20° C., 1013 mbar) between about 50° C. and about 110° C., preferably between about 70° C. and about 105° C. Ethanol, isopropanol and water have proven to be particularly suitable and for this reason are preferred as the polar solvent a1).

Especially preferred polar solvents a1) or solvent systems are characterized in that

the amount by weight of water in the total weight of the polar solvent a1) is more than about 80 wt %, preferably more than about 88 wt % and in particular more than about 92 wt %;

characterized in that the amount by weight of water and ethanol in the total weight of the polar solvent a1) is at least about 60 wt %, preferably at least about 80 wt %, especially preferably at least about 90 wt % and in particular at least about 95 wt %;

the amount by weight of water and ethanol in the total weight of the polar solvent a1) is at least about 60 wt %, preferably at least about 80 wt %, especially preferably at least about 90 wt % and in particular at least about 95 wt %, wherein the weight ratio of ethanol to water is preferably about 12:1 to about 4:1, preferably about 10:1 to about 6:1.

Another important ingredient of the cosmetic compositions as contemplated herein is cationic polymer a2). With respect to manufacturability, applicability and cosmetic effect of cosmetic compositions as contemplated herein, it has proven advantageous if the amount by weight of the cation polymer a2) to the total weight of the cationic preparation a) amounts to from about 0.2 to about 4.0 wt %, preferably about 0.5 to about 3.0 wt %.

The group of cationic polymers a3) comprises in particular the cationic polymers with the INCI designations polyquaternium-1, polyquaternium-2, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-22, polyquaternium-24, polyquaternium-27, polyquaternium-28, polyquaternium-29, polyquaternium-30, polyquaternium-31, polyquaternium-32, polyquaternium-33, polyquaternium-34, polyquaternium-35, polyquaternium-36, polyquaternium-39, polyquaternium-45, polyquaternium-46, polyquaternium-47, polyquaternium-48, polyquaternium-49, polyquaternium-50, polyquaternium-55, polyquaternium-56, polyquaternium-68 and polyquaternium-69. The most especially preferred cosmetic product is characterized in that the cationic polymer a2) is selected from the group of cationic polymers with the INCI designations polyquaternium-4, polyquaternium-11, polyquaternium-16, polyquaternium-55, polyquaternium-69. Corresponding cosmetic products are characterized by especially good hair cosmetic properties, in particular care properties and styling properties.

The cationic polymers a2) may be selected as contemplated herein from cationic, quaternized cellulose derivatives.

A first group of particularly preferred cationic polymers a2) includes the cationic cellulose derivatives, in particular those derived from a reaction of hydroxyethyl cellulose with a dimethyldiallyl ammonium reactant (in particular dimethyldiallyl ammonium chloride), optionally in the presence of additional reactants. Of these cationic celluloses, those cationic celluloses with the INCI designation polyquaternium-4 are again especially preferred. Corresponding celluloses are distributed by the National Starch Company, for example, under the brand names Celquat® H 100 and Celquat® L 200.

In addition, suitable cationic hair-setting polymers are those comprising at least one structural unit of the formula (M-I) and at least one structural unit of the formula (M-II) and optionally at least one structural unit of the formula (M-III):

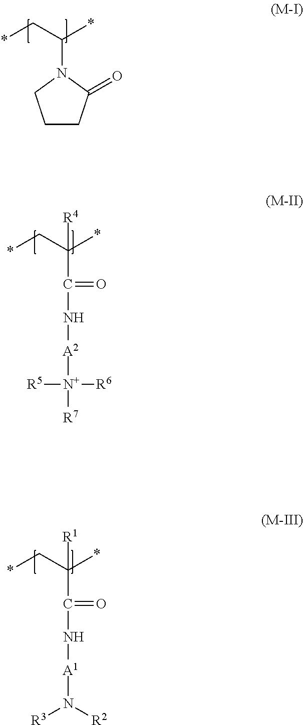

where
R¹ and R⁴ independently of one another stand for a hydrogen atom or a methyl group,
A¹ and A² independently of one another stand for a group comprising ethane-1,2-diyl, propane-1,3-diyl or butane-1,4-diyl,
R², R³, R⁵ and R⁶, independently of one another, stand for a (C₁ to C₄) alkyl group,
R⁷ stands for a (C₈ to C₃₀) alkyl group.

To compensate for the positive charge of this cationic polymer as well as all other cationic polymers, all the physiologically tolerable anions may be used such as, for example, chloride, bromide, hydrogen sulfate, methyl sulfate, ethyl sulfate, tetrafluoroborate, phosphate, hydrogen phosphate, dihydrogen phosphate, p-toluenesulfonate or triflate.

A second group of particularly preferred cationic polymers a2) includes the copolymers of vinylpyrrolidone with quaternized derivatives of dialkylaminoalkyl acrylate and methacrylate, in particular copolymers of vinylpyrrolidone and dimethylaminoethyl methacrylate (INCI designation polyquaternium-11) quaternized with diethyl sulfate. Corresponding polymers are available under the commercial designations Gafquat® 440, Gafquat® 734 and Gafquat® 755 N (ISP).

A third group of particularly preferred cationic polymers a2) preferably also include copolymers of methacryloylaminopropyllauryldimethyl ammonium chloride with vinylpyrrolidone and dimethylaminopropyl methacrylamide (INCI designation: polyquaternium-55), which are available from the company ISP under the brand names Styleze® W-10 or Styleze® W-20, for example.

Finally, a fourth group of particularly preferred cationic polymers a2) includes copolymers of N-vinylpyrrolidone, N-vinylcaprolactam, N-(3-dimethylaminopropyl)methacrylamide and 3-(methacryloylamino)propyllauryldimethyl ammonium chloride (INCI designation polyquaternium-69), which are distributed, for example, under the brand name AquaStyle® 300 (28-32 wt % active substance in an ethanol-water mixture, molecular weight 350,000) by the company ISP.

Additional preferred cationic polymers contain at least one structural element of the formula (M-IV):

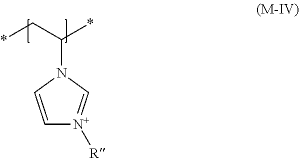

where
R⁻ stands for a (C₁ to C₄) alkyl group, in particular a methyl group
and additionally at least one other cationic and/or nonionic structural element.

It is again preferable if the cosmetic preparation a) contains at least one cationic copolymer comprising additionally a structurally element of the formula (M-I) in addition to at least one structural element of the formula (M-IV), as the cationic polymer a2):

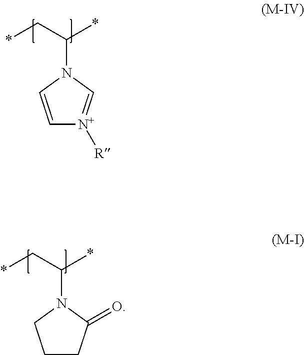

where
R″ stands for a (C₁ to C₄) alkyl group, in particular a methyl group. Most especially preferred cationic hair styling polymers contain from about 10 to about 30 mol %, preferably from about 15 to about 25 mol % and in particular about 20 mol % structural units according to formula (M-IV) and from about 70 to about 90 mol %, preferably from about 75 to about 85 mol % and in particular about 80 mol % structural units according to formula (M-I).
It is particularly preferred here if the cationic polymers a2) contain, in addition to polymer units resulting from the incorporation of the aforementioned structural units according to formula (M-IV) and (M-I) into the copolymer, max. about 5 wt %, preferably max. about 1 wt % polymer units that can be attributed to the incorporation of other monomers. The copolymers (c1) are preferably constructed exclusively from structural units of the formula (M-IV), where R″=methyl and (M-I) and can be described by the general formula (Poly1):

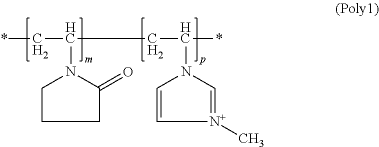

where the indices m and p each vary according to the molecular weight of the polymer and are not intended to denote that these are block copolymers. Instead, structural units of the formula (M-IV) and the formula (M-I) may be present in random distribution in the molecule.
If, in compensation for the positive charge of the polymer of formula (Poly1), a chloride ion is used, then these N-methylvinylimidazole/vinylpyrrolidone copolymers are identified as polyquaternium-16 according to INCI nomenclature.

A fifth group of especially preferred cationic polymers a2) comprises N-methylvinylimidazole/vinylpyrrolidone copolymers (INCI designation polyquaternium-16), which are distributed by BASF under the brand names Luviquat® Style, Luviquat® FC 370, Luviquat® FC 550, Luviquat® FC 905 and Luviquat® HM 552, for example.

If a methosulfate is used to compensate for the positive charge of the polymer of formula (Poly1), then these N-methylvinylimidazole/vinylpyrrolidone copolymers are designated as polyquaternium-44 according to the INCI nomenclature and are available from BASF under the brand name Luviquat® UltraCare, for example.

Additional preferred cationic polymers containing at least one structural unit according to the formula (M-IV-a) and at least one structural unit according to the formula (M-I) and at least one structural unit according to the formula (M-V):

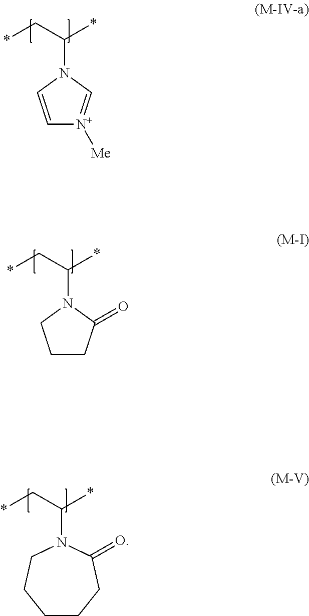

Here again, it is especially preferred within the scope of this embodiment if the copolymers contain, in addition to polymer units resulting from the incorporation of the aforementioned structural units according to formula (M-IV-a), (M-I) and (M-V) into the copolymer, max. 5 wt %, preferably max. 1 wt % polymer units attributed to the incorporation of other monomers. The copolymers (c2) are preferably constructed exclusively of the structural units of the formulas (M-IV-a), (M-I) and (M-V), and they are described by the general formula (Poly2)

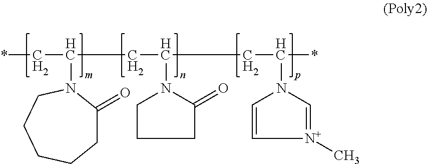

where the indices m, n and p each vary according to the molecular weight of the polymer and this should not mean that these are block copolymers. Instead, structural units of said formulas may be present in random distribution in the molecule.

If a methosulfate is used to compensate for the positive charge of the polymer of the formula (Poly2), then such N-methylvinylimidazole/vinylpyrrolidone/vinyl caprolactam copolymers are referred to as polyquaternium-46 according to the INCI nomenclature and are available from BASF under the brand name Luviquat® Hold, for example.

The most especially preferred copolymers of the formula (Poly2) contain from about 1 to about 20 mol %, preferably from about 5 to about 15 mol % and in particular about 10 mol % structural units according to the formula (M-IV-a) and from about 30 to about 50 mol %, preferably from about 35 to about 45 mol % and in particular 40 mol % structural units according to the formula (M-I) and from about 40 to about 60 mol %, preferably from about 45 to about 55 mol % and in particular about 60 mol % structural units according to the formula (M-V).

Additional preferred cationic polymers a2) contain at least one structural unit according to the formula (M-IV-a) and at least one additional structural unit according to formula (M-I) and at least one additional structural unit according to the formula (M-IV) and at least one additional structural unit according to the formula (M-VII)

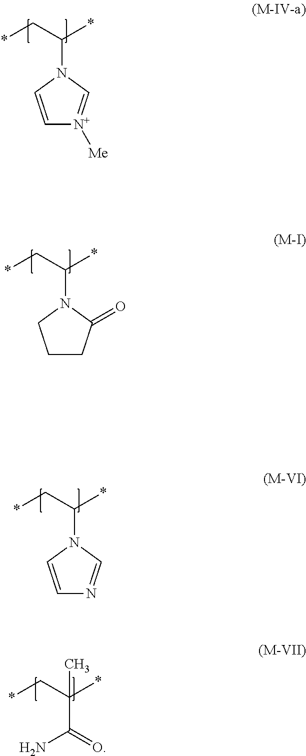

Here again, it is especially preferred within the scope of this embodiment if these copolymers contain max. 5 wt %, preferably max. 1 wt % polymers units attributed to the incorporation of other monomers, in addition to polymer units resulting from the incorporation of the aforementioned structural units according to the formulas (M-IV-a), (M-I), (M-VI) and (M-VII) into the copolymer. The copolymers are constructed exclusively from structural units of the formulas (M-IV-a), (M-I), (M-VI) and (M-VII) and can be described by the general formula (Poly3)

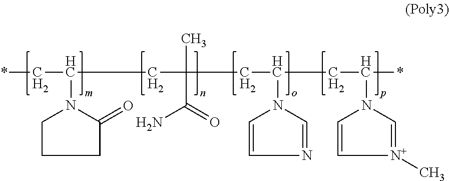

where the indices m, n, o and p each vary, depending on the molecular weight of the polymer, and this should not mean that they are block copolymers. Instead, structural units of the formulas (M-IV-a), (M-I), (M-VI) and (M-VII) may be present in the molecule in random distribution.

If a methosulfate is used to compensate for the positive charge of the polymer of the formula (Poly3), then such N-methylvinylimidazole/vinylpyrrolidone/vinyl caprolactam copolymers are referred to as polyquaternium-46 according to the INCI nomenclature and are available from BASF under the brand name Luviquat® Supreme, for example.

The most especially preferred copolymers (c3) contain from about 1 to about 12 mol %, preferably from about 3 to about 9 mol % and in particular about 6 mol % structural units according to the formula (M-IV-a) and from about 45 to 65 about mol %, preferably from about 50 to about 60 mol %, and in particular 55 mol % structural units according to the formula (M-I) and from about 1 to about 20 mol %, preferably from about 5 to about 15 mol % and in particular about 10 mol % structural units according to the formula (M-VI) and from about 20 to about 40 mol %, preferably from about 25 to about 35 mol % and in particular about 29 mol % structural units according to the formula (M-VII).

A third essential ingredient of cosmetic preparations a) as contemplated herein is the nonionic polymer a3). Nonionic polymers may be of synthetic or natural origin. Preferred cosmetic preparations a) contain, based on their total weight, from about 1.0 to about 10 wt %, preferably from about 2.0 to about 8.0 wt % nonionic polymer a3). This polymer a3) is different from the cationic polymer a2).

Based on their cosmetic effect in combination with the cationic polymer a2), nonionic polymers a3) that are especially preferred as contemplated herein are the vinylpyrrolidone homopolymers or copolymers. Especially preferred polymers a3) include, for example:

polyvinylpyrrolidones (INCI designation PVP), such as those distributed under the brand name Luviskol® (BASF),

vinylpyrrolidone/vinyl ester copolymers (INCI designation VP/VA copolymer) such as those distributed under the brand name Luviskol® (BASF). Preferred nonionic polymers include Luviskol® VA 64 and Luviskol® VA 73, both of which are vinylpyrrolidone/vinyl acetate copolymers.

Preferred cosmetic products are characterized in that the film-forming polymer a3) is selected from the group of nonionic polymers, preferably from the group of polyvinylpyrrolidones and vinylpyrrolidone/vinyl acetate copolymers, preferably vinylpyrrolidone/vinyl acetate copolymers.

Based on their better-than-average styling properties, cosmetic preparations a) containing at least two cationic polymers a2) as well as at least one nonionic polymer a3) are preferred. The combination of polyquatemium-4, polyquatemium-11 and a vinylpyrrolidone/vinyl acetate copolymer is especially preferred.

In addition to the cosmetic preparation a), the cosmetic products as contemplated herein additionally comprise a device for flash vaporization. The term “flash vaporization” within the context of the present patent application refers to the formation of vapor when the pressure drops in a closed space filled with liquid under an excess pressure. A corresponding excess pressure can be created, for example, by heating a quantity of the cosmetic preparation a) in a closed space to a temperature T₁. In the closed space, the liquid has a saturation pressure pi at a given temperature T₁. If the closed space is opened by employing a valve, for example, to a relaxation space at the pressure p₀<p₁, which is not under an excess pressure, then the pressure in the previously closed space drops and the cosmetic preparation a1) and/or the solvent contained in the preparation or portions of this solvent evaporate within the context of the propagation of a new pressure level. The resulting vapor or spray mist can be utilized for the application of specific cosmetic preparations.

If the cosmetic preparation a) is this heated in a closed space, starting from standard conditions (T₀=25° C., p₀=1.000 bar), the result is an elevated pressure of the cosmetic preparation a) in addition to an elevated temperature. This elevated pressure may be released in a relaxation space to a pressure p₀, for example, the ambient atmospheric pressure (p₀=1.000 bar), so that at least partial vaporization of the cosmetic preparation a) is achieved.

The cosmetic preparation a) can be depressurized directly in the space in which it was previously heated. Alternatively, however, the heated cosmetic preparation a), which is under an excess pressure, may also be transported, after being heated, into a second space, in which the pressure relaxation then takes place subsequently.

In other words, flash vaporization is a method in which the cosmetic preparation a) is heated in a closed container by employing a heating device to temperatures above the ambient temperature, wherein a pressure above ambient pressure develops in the container, and the cosmetic preparation a) that is heated and is under an elevated pressure is then released from the container into the surroundings.

A device for flash vaporization is thus a device comprising container and a heating device and designed, so that the cosmetic preparation a) can be heated to temperatures above ambient temperature in the closed container by employing the heating device in such a way that a pressure higher than ambient pressure is established in the container and the heated cosmetic preparation a), which is under an elevated pressure, can be released from the container into the surroundings.

Simultaneously with or after the pressure release, the cosmetic preparation a) may be sent to a nozzle, such that the properties of the vapor and/or spray mist created by the flash vaporization, in particular the droplet size or the droplet density or even the spray width and the shape of the spray cone can be influenced. The use of nozzles, preferably atomizer nozzles, is therefore preferred. The specific type of nozzle or the specific nozzle design is defined in a targeted manner as a function of the respective spray mist properties.

In summary, a preferred device for flash vaporization has
b1) a container b1) that is to be opened and closed by employing a valve and defines the closed interior space, in which the cosmetic preparation can be accommodated,
b2) a heating device b2), which makes it possible to heat a cosmetic preparation in the container b1).

The use of an additional nozzle b3), which permits atomization of the cosmetic preparation a) escaping from the container, is especially preferred. As an alternative to a valve, a closing element, which has a comparable effect and can open or close a respective opening in the container through a corresponding change in position, may also be used.

A preferred subject matter of the present disclosure is a cosmetic product comprising:
a) a cosmetic preparation containing, based on its total weight:
a1) from about 45 to about 99 wt % of at least one polar solvent;
a2) from about 0.1 to about 6.0 wt % cationic polymer;
a3) from about 0.1 to about 12 wt % nonionic polymer;
b) a device for flash vaporization of the cosmetic preparation a), wherein the device for flash vaporization comprises a container b1) and a heating device b2) and is designed so that:

the cosmetic preparation a) can be accommodated in the interior of the container b1),

the interior of the container b1), which is at least partially filled with the cosmetic preparation a), can be sealed,

the cosmetic preparation a) can be heated in the closed interior of the container b1) by employing the heating device b2) with an increase in pressure.

An especially preferred subject matter of the present disclosure is a cosmetic product comprising
a) a cosmetic preparation containing, based on its total weight:
a1) from about 45 to about 99 wt % of at least one polar solvent;
a2) from about 0.1 to about 6.0 wt % cationic polymer;
a3) from about 0.1 to about 12 wt % nonionic polymer;
b) a device for flash vaporization of the cosmetic preparation a), comprising
b1) a container b1) to be opened and closed by employing a valve;
b2) a heating device, which makes it possible to heat a cosmetic preparation in the container b1);
b3) a nozzle b3), which makes it possible to atomize the cosmetic preparation a).

Another preferred subject matter of the present disclosure is a cosmetic product comprising:
a) a cosmetic preparation containing, based on its total weight:
a1) from about 45 to about 99 wt % of at least one polar solvent, wherein the amount by weight of water in the total weight of the polar solvent a1) is preferably at least about 80 wt %, especially at least about 88 wt %, especially preferably at least about 92 wt %,
a2) from about 0.1 to about 6.0 wt % cationic polymer;
a3) from about 0.1 to about 12 wt % nonionic polymer;
b) a device for flash vaporization of the cosmetic preparation a), comprising
b1) a container b1) to be opened and closed by employing a valve;
b2) a heating device, which makes it possible to heat a cosmetic preparation in the container b1);
b3) a nozzle b3), which makes it possible to atomize cosmetic preparation a).

In other words, an especially preferred subject matter of the present disclosure is a cosmetic product, comprising:
a) a cosmetic preparation containing, based on its total weight:
a1) from about 45 to about 99 wt % of at least one polar solvent;
a2) from about 0.1 to about 6.0 wt % cationic polymer;
a3) from about 0.1 to about 12 wt % nonionic polymer;
b) a device for flash vaporization of the cosmetic preparation a), wherein the device for flash vaporization comprises a container b1) and a heating device b2) and is designed so that:

the cosmetic preparation a) can be accommodated in the interior of the container b1),

the interior of the container b1), which is at least partially filled with the cosmetic preparation a), can be sealed,

the cosmetic preparation a) can be heated in the closed interior of the container b1) by employing the heating device b2) with an increase in pressure,

the heated cosmetic preparation a) can be depressurized from the interior of container b1) into the surroundings with a reduction in pressure.

Container b1), in which the cosmetic preparation is heated, is designed to make it possible to completely seal this container from the surroundings during the heating of the cosmetic preparation a), to enable flash vaporization of the cosmetic preparation a). This can be ensured by a flow control component, for example, in particular a valve.

The container b1), in which the cosmetic preparation is heated, is preferably in contact with another container, from which the amount of cosmetic preparation provided for flash vaporization is transferred into the container b1) before heating. The access between this storage container and container b1) is via a corresponding device, for example, a valve that is to be opened and closed. This additional container is preferably designed in the form of a storage container, i.e., preferably comprising several times, for example, more than about 10 times, preferably more than about 50 times the amount of cosmetic preparation required for a vaporization process. In other words, the additional container/storage container preferably has a volume more than about 10 times greater, preferably more than about 20 times greater and in particular more than about 50 times greater than that of container b1).

Another especially preferred subject matter of the present disclosure is a cosmetic product comprising:
a) a cosmetic preparation containing, based on its total weight:
a1) from about 45 to about 99 wt % of at least one polar solvent;
a2) from about 0.1 to about 6.0 wt % cationic polymer;
a3) from about 0.1 to about 12 wt % nonionic polymer;
b) a device for flash vaporization of the cosmetic preparation a), comprising
b1) a container b1) to be opened and closed by employing a valve;
b2) a heating device, which makes it possible to heat a cosmetic preparation in the container b1)
b3) a nozzle b3), which makes it possible to atomize the cosmetic preparation a),
c) a storage container for the cosmetic preparation a), from which the cosmetic preparation a) can enter the container b1), wherein

the access between the storage container and container b1) has a flow control component such that the flow of the cosmetic preparation a) out of the storage container and into the container b1) can be interrupted;

the storage container has a volume at least about 10 times, preferably at least about 20 times and in particular at least about 50 times greater than the volume of container 1)1).

The storage container is not a pressurized container and the cosmetic composition in the storage container is not under pressure. In other words, the pressure in the interior of the storage container corresponds to the ambient pressure (also referred to as the atmospheric pressure or air pressure). Thus, the corresponding cosmetic products, for example, do not include a propellant. The cosmetic product does not have a pump device suitable for releasing or spraying the cosmetic preparation into the surroundings without the influence of the device for flash vaporization.

A most especially preferred subject matter of the present disclosure is a cosmetic product comprising:
a) a cosmetic preparation containing, based on its total weight:
a1) from about 45 to about 99 wt % of at least one polar solvent;
a2) from about 0.1 to about 6.0 wt % cationic polymer;
a3) from about 0.1 to about 12 wt % nonionic polymer;
b) a device for flash vaporization of the cosmetic preparation a), comprising:
b1) a container b1) to be opened and closed by employing a valve
b2) a heating device, which makes it possible to heat a cosmetic preparation in the container b1);
b3) a nozzle b3), which makes it possible to atomize the cosmetic preparation a);
c) a storage container for the cosmetic preparation a), from which the cosmetic preparation a) can enter the container b1), wherein

the access between the storage container and container b1) has a flow control component, such that the flow of the cosmetic preparation a) out of the storage container and into the container b1) can be interrupted;

the storage container has a volume at least 10 times, preferably at least about 50 times greater than the volume of container b1),

the pressure in the interior of the storage container corresponds to ambient pressure.

A most especially preferred subject matter of the present disclosure is a cosmetic product comprising:
a) a cosmetic preparation containing, based on its total weight:
a1) from about 45 to about 99 wt % of at least one polar solvent;
a2) from about 0.1 to about 6.0 wt % cationic polymer;
a3) from about 0.1 to about 12 wt % nonionic polymer;
b) a device for flash vaporization of the cosmetic preparation a), comprising:
b1) a container b1) to be opened and closed by employing a valve;
b2) a heating device, which makes it possible to heat a cosmetic preparation in the container b1);
b3) a nozzle b3), which makes it possible to atomize the cosmetic preparation a);
c) a storage container for the cosmetic preparation a), from which the cosmetic preparation a) can enter the container b1), wherein

the access between the storage container and container b1) has a flow control component, such that the flow of the cosmetic preparation a) out of the storage container and into the container b1) can be interrupted;

the storage container has a volume at least about 10 times, preferably at least about 50 times greater than the volume of container b1),

the pressure in the interior of the storage container corresponds to ambient pressure, and the cosmetic product does not contain a propellant.

Also preferred are cosmetic products, comprising:
a) a cosmetic preparation containing, based on its total weight:
a1) from about 45 to about 99 wt % of at least one polar solvent;
a2) from about 0.1 to about 6.0 wt % cationic polymer;
a3) from about 0.1 to about 12 wt % nonionic polymer;
b) a device for flash vaporization of the cosmetic preparation a), comprising:
b1) a container b1) to be opened and closed by employing a valve
b2) a heating device, which makes it possible to heat a cosmetic preparation in the container b1)
b3) a nozzle b3), which makes it possible to atomize the cosmetic preparation a),
c) a storage container for the cosmetic preparation a), from which the cosmetic preparation a) can enter the container b1), wherein

the access between the storage container and container b1) has a flow control component, such that the flow of the cosmetic preparation a) out of the storage container and into the container b1) can be interrupted;

the storage container has a volume at least about 10 times, preferably at least about 50 times greater than the volume of container b1),

the pressure in the interior of the storage container corresponds to ambient pressure, wherein the cosmetic product does not have a pump device suitable for releasing or spraying the cosmetic preparation a) into the surroundings without the influence of the device for flash vaporization.

In summary, a particularly preferred subject matter of the present disclosure is therefore a cosmetic product, comprising:
a) a cosmetic preparation containing, based on its total weight:
a1) 45 to about 99 wt % of at least one polar solvent;
a2) 0.1 to about 6.0 wt % cationic polymer;
a3) 0.1 to about 12 wt % nonionic polymer;
b) a device for flash vaporization of the cosmetic preparation a), comprising
b1) a container b1) to be opened and closed by employing a valve
b2) a heating device, which makes it possible to heat a cosmetic preparation in the container b1)
b3) a nozzle b3), which makes it possible to atomize the cosmetic preparation a),
c) a storage container for the cosmetic preparation a), out of which the cosmetic preparation a) can enter the container b1), wherein

the access between the storage container and container b1) has a flow control component such that the flow of the cosmetic preparation a) out of the storage container and into the container b1) can be interrupted;

the storage container has a volume at least 10 times, preferably at least about 50 times greater than the volume of container b1),

the pressure in the interior of the storage container corresponds to ambient pressure and the cosmetic product does not contain a propellant,

wherein the cosmetic product does not have a pump device suitable for releasing or spraying the cosmetic preparation a) into the surroundings without the influence of the device for flash vaporization.

In addition to the two ingredients a1), a2) and a3) described above, the cosmetic preparations a) as contemplated herein may contain additional active ingredients or additives, but in particular those active ingredients or additives that improve the manufacturability, applicability and/or cosmetic effect of the cosmetic preparations as contemplated herein are preferred.

To improve the manufacturability, applicability and cosmetic effect, the cosmetic preparation a) preferably contains nonionic surfactants a4), wherein especially preferred cosmetic preparations a) are characterized in that they contain, based on their total weight, from about 0.02 to about 4.0 wt %, preferably from about 0.05 to about 2.0 wt % and in particular from about 0.1 to about 1.0 wt % nonionic surfactant a4).

Preferred nonionic surfactants include PEG derivatives of hydrogenated castor oil such as those available under the designation PEG hydrogenated castor oil, e.g., PEG-30 hydrogenated castor oil, PEG-33 hydrogenated castor oil, PEG-35 hydrogenated castor oil, PEG-36 hydrogenated castor oil, PEG-40 hydrogenated castor oil or PEG-60 hydrogenated castor oil. As contemplated herein, nonionic surfactants are especially preferably selected from the group of PEG derivatives of hydrogenated castor oil, especially preferably from the group of PEG-40 hydrogenated castor oil and PEG-60 hydrogenated castor oil, in particular PEG-40 hydrogenated castor oil.

Another preferred ingredient of cosmetic preparations a) as contemplated herein is cationic surfactants a5). Preferred cationic surfactants a5) are selected from quaternary ammonium compounds, esterquats and amidoamines. Cationic surfactants are contained in the cosmetic preparation a) in amounts of from about 0.05 to about 3.0 wt %, preferably from about 0.1 to about 2.0 wt % and in particular from about 0.2 to about 1.0 wt %, based on their total weight. Cationic surfactants a5) from the group of quaternary ammonium compounds are especially preferred.

Preferred quaternary ammonium compounds include ammonium halides in particular chlorides and bromides such as alkyltrimethyl ammonium chlorides, dialkyldimethyl ammonium chlorides and trialkylmethyl ammonium chlorides, for example cetyltrimethyl ammonium chloride, stearyltrimethyl ammonium chloride, distearyldimethyl ammonium chloride, lauryldimethyl ammonium chloride, lauryldimethyl ammonium chloride, lauryldimethylbenzyl ammonium chloride and tricetylmethyl ammonium chloride, as well as the imidazolium compounds known under the INCI designations quaternium-27 and quaternium-83. The long alkyl chains of the aforementioned surfactants preferably contain 10 to 18 carbon atoms. Most especially preferred cosmetic compositions a) as contemplated herein are characterized in that they contain, based on their total weight, from about 0.05 to about 3.0 wt %, preferably from about 0.1 to about 2.0 wt % and in particular from about 0.2 to about 1.0 wt % (C₁₂ to C₁₈) alkyltrimethyl ammonium salt(s).

Other suitable active ingredients or additives include in particular additional care substances. Examples of care substances that may be contained in the agent include a protein hydrolyzate and/or one of its derivatives. Protein hydrolyzates are product mixtures obtained by acidic, basic or enzymatically catalyzed degradation of proteins. The term protein hydrolyzates is also understood as contemplated herein to include total hydrolyzates as well as individual amino acids and their derivatives as well as mixtures of various amino acids. The molecular weight of the protein hydrolyzates that can be used as contemplated herein is between about 75 Dalton, the molecular weight for glycine, and about 200,000 Dalton. The molecular weight is preferably about 75 to about 50,000 Dalton and most especially preferably from about 75 to about 20,000 Dalton.

The agent as contemplated herein may additionally contain as the care substance a vitamin, a provitamins, a vitamin precursor and/or one of their derivatives. As contemplated herein, the preferred vitamin, provitamins and vitamin precursors are those usually assigned to the groups A, B, C, E, F and H.

Additional care substances include panthenol, caffeine, nicotinamide and sorbitol.

The agents as contemplated herein may additionally contain at least one plant extract as the care substance, but mono- and/or oligosaccharides and/or lipids may also be present.

The composition of some especially preferred cosmetic preparations as contemplated herein can be found in the following tables (amounts given in wt %, based on the total weight of the cosmetic agent, unless otherwise indicated). With respect to additional preferred embodiments of these particularly preferred compositions, what was said above regarding the cosmetic preparations a) as contemplated herein also applies here mutatis mutandis.

| | Formula | Formula | Formula | Formula | Formula |
|---|---|---|---|---|---|
| | 1 | 2 | 3 | 4 | 5 |
| Polar solvent a1) | 45 to 99 | 70 to 98 | 80 to 95 | 94 | 90 |
| Cationic polymer | 0.1 to 6.0 | 0.2 to 4.0 | 0.5 to 3.0 | 2.3 | 2.0 |
| a2) | | | | | |
| Nonionic polymer | 0.1 to 12 | 1.0 to 10 | 2.0 to 8.0 | 2.1 | 5.5 |
| a3) | | | | | |
| Optional additives | to 100 | to 100 | to 100 | to 100 | to 100 |

| | Formula | Formula | Formula | Formula | Formula |
|---|---|---|---|---|---|
| | 6 | 7 | 8 | 9 | 10 |
| Polar solvent a1) | 45 to 99 | 70 to 98 | 80 to 95 | 94 | 90 |
| Polyquaternium-4 | 0.1 to 6.0 | 0.2 to 4.0 | 0.5 to 3.0 | 2.3 | 2.0 |
| Nonionic polymer | 0.1 to 12 | 1.0 to 10 | 2.0 to 8.0 | 2.1 | 5.5 |
| a3) | | | | | |
| Optional additives | to 100 | to 100 | to 100 | to 100 | to 100 |

| | Formula | Formula | Formula | Formula | Formula |
|---|---|---|---|---|---|
| | 11 | 12 | 13 | 14 | 15 |
| Polar solvent a1) | 45 to 99 | 70 to 98 | 80 to 95 | 94 | 90 |
| Polyquaternium- | 0.1 to 6.0 | 0.2 to 4.0 | 0.5 to 3.0 | 2.3 | 2.0 |
| 11 | | | | | |
| Nonionic polymer | 0.1 to 12 | 1.0 to 10 | 2.0 to 8.0 | 2.1 | 5.5 |
| a3) | | | | | |
| Optional additives | to 100 | to 100 | to 100 | to 100 | to 100 |

| | Formula | Formula | Formula | Formula | Formula |
|---|---|---|---|---|---|
| | 16 | 17 | 18 | 19 | 20 |
| Polar solvent a1) | 45 to 99 | 70 to 98 | 80 to 95 | 94 | 90 |
| Polyquaternium- | 0.1 to 6.0 | 0.2 to 4.0 | 0.5 to 3.0 | 2.3 | 2.0 |
| 16 | | | | | |
| Nonionic polymer | 0.1 to 12 | 1.0 to 10 | 2.0 to 8.0 | 2.1 | 5.5 |
| a3) | | | | | |
| Optional additives | to 100 | to 100 | to 100 | to 100 | to 100 |

| | Formula | Formula | Formula | Formula | Formula |
|---|---|---|---|---|---|
| | 21 | 22 | 23 | 24 | 25 |
| Polar solvent a1) | 45 to 99 | 70 to 98 | 80 to 95 | 94 | 90 |
| Polyquaternium- | 0.1 to 6.0 | 0.2 to 4.0 | 0.5 to 3.0 | 2.3 | 2.0 |
| 55 | | | | | |
| Nonionic polymer | 0.1 to 12 | 1.0 to 10 | 2.0 to 8.0 | 2.1 | 5.5 |
| a3) | | | | | |
| Optional additives | to 100 | to 100 | to 100 | to 100 | to 100 |

| | Formula | Formula | Formula | Formula | Formula |
|---|---|---|---|---|---|
| | 26 | 27 | 28 | 29 | 30 |
| Polar solvent a1) | 45 to 99 | 70 to 98 | 80 to 95 | 94 | 90 |
| Polyquaternium- | 0.1 to 6.0 | 0.2 to 4.0 | 0.5 to 3.0 | 2.3 | 2.0 |
| 69 | | | | | |
| Nonionic polymer | 0.1 to 12 | 1.0 to 10 | 2.0 to 8.0 | 2.1 | 5.5 |
| a3) | | | | | |
| Optional additives | to 100 | to 100 | to 100 | to 100 | to 100 |

| | Formula | Formula | Formula | Formula | Formula |
|---|---|---|---|---|---|
| | 31 | 32 | 33 | 34 | 35 |
| Polar solvent a1) | 45 to 99 | 70 to 98 | 80 to 95 | 94 | 90 |
| Cationic polymer | 0.1 to 6.0 | 0.2 to 4.0 | 0.5 to 3.0 | 2.3 | 2.0 |
| a2) | | | | | |
| Vinylpyrrolidone/ | 0.1 to 12 | 1.0 to 10 | 2.0 to 8.0 | 2.1 | 5.5 |
| vinyl acetate | | | | | |
| copolymer | | | | | |
| Optional additives | to 100 | to 100 | to 100 | to 100 | to 100 |

| | Formula | Formula | Formula | Formula | Formula |
|---|---|---|---|---|---|
| | 36 | 37 | 38 | 39 | 40 |
| Polar solvent | 45 to 99 | 70 to 98 | 80 to 95 | 94 | 90 |
| a1) | | | | | |
| Cationic | 0.1 to 6.0 | 0.2 to 4.0 | 0.5 to 3.0 | 2.3 | 2.0 |
| polymer a2) | | | | | |
| Nonionic | 0.1 to 12 | 1.0 to 10 | 2.0 to 8.0 | 2.1 | 5.5 |
| polymer a3) | | | | | |
| Nonionic | 0.02 to 4.0 | 0.05 to 2.0 | 0.1 to 1.0 | 0.2 | 0.3 |
| surfactant | | | | | |
| a4) | | | | | |
| Optional | to 100 | to 100 | to 100 | to 100 | to 100 |
| additives | | | | | |

| | Formula | Formula | Formula | Formula | Formula |
|---|---|---|---|---|---|
| | 41 | 42 | 43 | 44 | 45 |
| Polar solvent | 45 to 99 | 70 to 98 | 80 to 95 | 94 | 90 |
| a1) | | | | | |
| Cationic | 0.1 to 6.0 | 0.2 to 4.0 | 0.5 to 3.0 | 2.3 | 2.0 |
| polymer a2) | | | | | |
| Nonionic | 0.1 to 12 | 1.0 to 10 | 2.0 to 8.0 | 2.1 | 5.5 |
| polymer a3) | | | | | |
| Cationic | 0.05 to 3.0 | 0.1 to 2.0 | 0.2 to 1.0 | 0.3 | 0.5 |
| surfactant a5) | | | | | |
| Optional | to 100 | to 100 | to 100 | to 100 | to 100 |
| additives | | | | | |

| | Formula | Formula | Formula | Formula | Formula |
|---|---|---|---|---|---|
| | 46 | 47 | 48 | 49 | 50 |
| Polar solvent a1) | 45 to 99 | 70 to 98 | 80 to 95 | 94 | 90 |
| Cationic | 0.1 to 6.0 | 0.2 to 4.0 | 0.5 to 3.0 | 2.3 | 2.0 |
| polymer a2) | | | | | |
| Nonionic | 0.1 to 12 | 1.0 to 10 | 2.0 to 8.0 | 2.1 | 5.5 |
| polymer a3) | | | | | |
| Nonionic) | 0.02 to 4.0 | 0.05 to 2.0 | 0.1 to 1.0 | 0.2 | 0.3 |
| surfactant a4 | | | | | |
| Cationic | 0.05 to 3.0 | 0.1 to 2.0 | 0.2 to 1.0 | 0.3 | 0.5 |
| surfactant a5) | | | | | |
| Optional | to 100 | to 100 | to 100 | to 100 | to 100 |
| additives | | | | | |

| | Formula | Formula | Formula | Formula | Formula |
|---|---|---|---|---|---|
| | 51 | 52 | 53 | 54 | 55 |
| Water | 45 to 99 | 70 to 98 | 80 to 95 | 94 | 90 |
| Cationic polymer | 0.1 to 6.0 | 0.2 to 4.0 | 0.5 to 3.0 | 2.3 | 2.0 |
| a2) | | | | | |
| Nonionic polymer | 0.1 to 12 | 1.0 to 10 | 2.0 to 8.0 | 2.1 | 5.5 |
| a3) | | | | | |
| Optional additives | to 100 | to 100 | to 100 | to 100 | to 100 |

| | Formula | Formula | Formula | Formula | Formula |
|---|---|---|---|---|---|
| | 56 | 57 | 58 | 59 | 60 |
| Water | 45 to 99 | 70 to 98 | 80 to 95 | 94 | 90 |
| Polyquaternium-4 | 0.1 to 6.0 | 0.2 to 4.0 | 0.5 to 3.0 | 2.3 | 2.0 |
| Nonionic polymer | 0.1 to 12 | 1.0 to 10 | 2.0 to 8.0 | 2.1 | 5.5 |
| a3) | | | | | |
| Optional additives | to 100 | to 100 | to 100 | to 100 | to 100 |

| | Formula | Formula | Formula | Formula | Formula |
|---|---|---|---|---|---|
| | 61 | 62 | 63 | 64 | 65 |
| Water | 45 to 99 | 70 to 98 | 80 to 95 | 94 | 90 |
| Polyquaternium- | 0.1 to 6.0 | 0.2 to 4.0 | 0.5 to 3.0 | 2.3 | 2.0 |
| 11 | | | | | |
| Nonionic polymer | 0.1 to 12 | 1.0 to 10 | 2.0 to 8.0 | 2.1 | 5.5 |
| a3) | | | | | |
| Optional additives | to 100 | to 100 | to 100 | to 100 | to 100 |

| | Formula | Formula | Formula | Formula | Formula |
|---|---|---|---|---|---|
| | 66 | 67 | 68 | 69 | 70 |
| Water | 45 to 99 | 70 to 98 | 80 to 95 | 94 | 90 |
| Polyquaternium- | 0.1 to 6.0 | 0.2 to 4.0 | 0.5 to 3.0 | 2.3 | 2.0 |
| 16 | | | | | |
| Nonionic polymer | 0.1 to 12 | 1.0 to 10 | 2.0 to 8.0 | 2.1 | 5.5 |
| a3) | | | | | |
| Optional additives | to 100 | to 100 | to 100 | to 100 | to 100 |

| | Formula | Formula | Formula | Formula | Formula |
|---|---|---|---|---|---|
| | 71 | 72 | 73 | 74 | 75 |
| Water | 45 to 99 | 70 to 98 | 80 to 95 | 94 | 90 |
| Polyquaternium- | 0.1 to 6.0 | 0.2 to 4.0 | 0.5 to 3.0 | 2.3 | 2.0 |
| 55 | | | | | |
| Nonionic polymer | 0.1 to 12 | 1.0 to 10 | 2.0 to 8.0 | 2.1 | 5.5 |
| a3) | | | | | |
| Optional additives | to 100 | to 100 | to 100 | to 100 | to 100 |

| | Formula | Formula | Formula | Formula | Formula |
|---|---|---|---|---|---|
| | 76 | 77 | 78 | 79 | 80 |
| Water | 45 to 99 | 70 to 98 | 80 to 95 | 94 | 90 |
| Polyquaternium- | 0.1 to 6.0 | 0.2 to 4.0 | 0.5 to 3.0 | 2.3 | 2.0 |
| 69 | | | | | |
| Nonionic polymer | 0.1 to 12 | 1.0 to 10 | 2.0 to 8.0 | 2.1 | 5.5 |
| a3) | | | | | |
| Optional additives | to 100 | to 100 | to 100 | to 100 | to 100 |

| | Formula | Formula | Formula | Formula | Formula |
|---|---|---|---|---|---|
| | 81 | 82 | 83 | 84 | 85 |
| Water | 45 to 99 | 70 to 98 | 80 to 95 | 94 | 90 |
| Cationic polymer | 0.1 to 6.0 | 0.2 to 4.0 | 0.5 to 3.0 | 2.3 | 2.0 |
| a2) | | | | | |
| Vinylpyrrolidone/ | 0.1 to 12 | 1.0 to 10 | 2.0 to 8.0 | 2.1 | 5.5 |
| vinyl acetate | | | | | |
| copolymer | | | | | |
| Optional additives | to 100 | to 100 | to 100 | to 100 | to 100 |

| | Formula | Formula | Formula | Formula | Formula |
|---|---|---|---|---|---|
| | 86 | 87 | 88 | 89 | 90 |
| Water | 45 to 99 | 70 to 98 | 80 to 95 | 94 | 90 |
| Cationic | 0.1 to 6.0 | 0.2 to 4.0 | 0.5 to 3.0 | 2.3 | 2.0 |
| polymer a2) | | | | | |
| Nonionic | 0.1 to 12 | 1.0 to 10 | 2.0 to 8.0 | 2.1 | 5.5 |
| polymer a3) | | | | | |
| Nonionic | 0.02 to 4.0 | 0.05 to 2.0 | 0.1 to 1.0 | 0.2 | 0.3 |
| surfactant | | | | | |
| a4) | | | | | |
| Optional | to 100 | to 100 | to 100 | to 100 | to 100 |
| additives | | | | | |

| | Formula | Formula | Formula | Formula | Formula |
|---|---|---|---|---|---|
| | 91 | 92 | 93 | 94 | 95 |
| Water | 45 to 99 | 70 to 98 | 80 to 95 | 94 | 90 |
| Cationic polymer | 0.1 to 6.0 | 0.2 to 4.0 | 0.5 to 3.0 | 2.3 | 2.0 |
| a2) | | | | | |
| Nonionic polymer | 0.1 to 12 | 1.0 to 10 | 2.0 to 8.0 | 2.1 | 5.5 |
| a3) | | | | | |
| Cationic | 0.05 to 3.0 | 0.1 to 2.0 | 0.2 to 1.0 | 0.3 | 0.5 |
| surfactant a5) | | | | | |
| Optional additives | to 100 | to 100 | to 100 | to 100 | to 100 |

| | Formula | Formula | Formula | Formula | Formula |
|---|---|---|---|---|---|
| | 96 | 97 | 98 | 99 | 100 |
| Water | 45 to 99 | 70 to 98 | 80 to 95 | 94 | 90 |
| Cationic | 0.1 to 6.0 | 0.2 to 4.0 | 0.5 to 3.0 | 2.3 | 2.0 |
| polymer a2) | | | | | |
| Nonionic | 0.1 to 12 | 1.0 to 10 | 2.0 to 8.0 | 2.1 | 5.5 |
| polymer a3) | | | | | |
| Nonionic | 0.02 to 4.0 | 0.05 to 2.0 | 0.1 to 1.0 | 0.2 | 0.3 |
| surfactant a4) | | | | | |
| Cationic | 0.05 to 3.0 | 0.1 to 2.0 | 0.2 to 1.0 | 0.3 | 0.5 |
| surfactant a5) | | | | | |
| Optional | to 100 | to 100 | to 100 | to 100 | to 100 |
| additives | | | | | |

| | Formula | Formula | Formula | Formula | Formula |
|---|---|---|---|---|---|
| | 101 | 102 | 103 | 104 | 105 |
| Water | 45 to 99 | 70 to 98 | 80 to 95 | 94 | 90 |
| Poly- | 0.1 to 6.0 | 0.2 to 4.0 | 0.5 to 3.0 | 2.3 | 2.0 |
| quaternium-4 | | | | | |
| Vinyl- | 0.1 to 12 | 1.0 to 10 | 2.0 to 8.0 | 2.1 | 5.5 |
| pyrrolidone/ | | | | | |
| vinyl acetate | | | | | |
| copolymer | | | | | |
| PEG-40 | 0.02 to 4.0 | 0.05 to 2.0 | 0.1 to 1.0 | 0.2 | 0.3 |
| hydrogenated | | | | | |
| castor oil | | | | | |
| Cetrimonium | 0.05 to 3.0 | 0.1 to 2.0 | 0.2 to 1.0 | 0.3 | 0.5 |
| chloride | | | | | |
| Optional | to 100 | to 100 | to 100 | to 100 | to 100 |
| additives | | | | | |

| | Formula | Formula | Formula | Formula | Formula |
|---|---|---|---|---|---|
| | 106 | 107 | 108 | 109 | 110 |
| Water | 45 to 99 | 70 to 98 | 80 to 95 | 94 | 90 |
| Combination of | 0.1 to 6.0 | 0.2 to 4.0 | 0.5 to 3.0 | 2.3 | 2.0 |
| poly- | | | | | |
| quaternium-4 | | | | | |
| and poly- | | | | | |
| quaternium-11 | | | | | |
| Vinyl- | 0.1 to 12 | 1.0 to 10 | 2.0 to 8.0 | 2.1 | 5.5 |
| pyrrolidone/ | | | | | |
| vinyl acetate | | | | | |
| copolymer | | | | | |
| PEG-40 | 0.02 to 4.0 | 0.05 to 2.0 | 0.1 to 1.0 | 0.2 | 0.3 |
| hydrogenated | | | | | |
| castor oil | | | | | |
| Cetrimonium | 0.05 to 3.0 | 0.1 to 2.0 | 0.2 to 1.0 | 0.3 | 0.5 |
| chloride | | | | | |
| Optional | to 100 | to 100 | to 100 | to 100 | to 100 |
| additives | | | | | |

Because of the ease with which they can be produced and their good cosmetic effect, the most preferred cosmetic preparations contain only small amounts of additional active ingredients and additives in addition to the ingredients a1) through a5) described above. Cosmetic preparations that are characterized in that the amount by weight of the ingredients a1), a2) and, if present, a3) and/or a4) and/or a5), relative to the total weight of the cosmetic preparation, amounts to at least about 80 wt %, preferably at least about 90 wt %, especially preferably at least about 93 wt % and in particular at least about 97 wt %. The most preferred cosmetic preparations comprise, based on their total weight, at least about 80 wt %, preferably at least about 87 wt % and in particular at least about 95 wt % of the ingredients a1), a2) and a3).

As stated in the introduction, the cosmetic preparations a) as contemplated herein are particularly suitable for application by employing a device for flash vaporization. Another subject matter of the present disclosure is therefore the use of a cosmetic preparation
a), containing, based on its total weight
a1) from about 45 to about 99 wt % of at least one polar solvent;
a2) from about 0.1 to about 6.0 wt % cationic polymer;
a3) from about 0.1 to about 12 wt % nonionic polymer
as the process material in a device for flash vaporization.

The subject matter of the present patent application is thus the use of a product as contemplated herein for treatment of keratin-containing fibers, in particular human hair, with a cosmetic preparation a) and/or for temporary shaping of keratin-containing fibers, in particular human hair.

Another subject matter of the present patent application is a method for temporary shaping of keratin-containing fibers, in particular human hair, in which the keratinic fibers are treated with a cosmetic preparation a) by employing a device for flash vaporization, said preparation containing, based on its total weight:
a1) from about 45 to about 99 wt % of at least one polar solvent;
a2) from about 0.1 to about 6.0 wt % cationic polymer;
a3) from about 0.1 to about 12 wt % nonionic polymer.

The cosmetic preparation a) is preferably converted by employing the device for flash vaporization to a spray mist, which is then used to treat the keratin-containing fibers.

To achieve an adequate spray effect, the cosmetic preparation a) is preferably heated to temperatures above the boiling point of the polar solvent or solvent mixture contained in the cosmetic preparation a).

If the polar solvent is water or a solvent mixture with a water content above about 50 wt % (based on the total weight of the solvent mixture), then the cosmetic preparation is preferably heated to temperatures above about 100° C., preferably to temperatures of about 100° C. and about 240° C., especially preferably to temperatures of about 140° C. to about 160° C.

The excess pressure achieved by heating the cosmetic preparation a) preferably amounts to between about 1.1 and about 8 bar, especially between about 1.2 and about 4 bar in those cases in which the polar solvent is water or a solvent mixture having a water content of more than about 50 wt % (based on the total weight of the solvent mixture).

A preferred subject matter of the patent application is a method for changing the color of keratin-containing fibers, in particular human hair, in which the keratin-containing fibers are treated, by employing a device for flash vaporization, with a cosmetic preparation a) containing, based on its total weight:
a1) from about 45 to about 99 wt % of at least one polar solvent;
a2) from about 0.1 to about 6.0 wt % cationic polymer;
a3) from about 0.1 to about 12 wt % nonionic polymer;
wherein

a partial quantity of the cosmetic preparation a) which is in the storage container is transferred to a container b1) from said storage container, in the interior of which a pressure corresponding to the ambient pressure prevails;

then the access between the storage container and container b1) is interrupted by a flow control component, such that the flow of cosmetic preparation a) out of the storage container and into container b1) can be interrupted;

then the cosmetic preparation a) that is in the container b1), which is sealed with respect to the environment, is heated by employing a heating device, so that the pressure in the interior of container b1) rises to levels above ambient pressure, preferably to levels between about 1.1 and about 8 bar, in particular to levels between about 1.2 and about 4 bar;

then the container b1), which is under a higher pressure than ambient pressure, is opened in such a manner that it depressurizes the outlet of at least a partial amount, preferably at least about 50 wt %, especially at least about 80 wt % and in particular at least about 90 wt % of the cosmetic preparation in container b1) is released out of container b1) and into the surroundings with a reduction in the pressure prevailing in the container b1) at the point in time of the opening of the container.

Release of the cosmetic preparation a) into the surroundings preferably takes place with the development of a spray mist of the cosmetic preparation a).

The cosmetic preparation a) released from the container b1) is preferably applied to keratinic fibers, in particular human hair.

Methods in the course of which the cosmetic preparation released from the container b1) is passed through a nozzle before treatment of the keratinic fibers are especially preferred.

With respect to other preferred embodiments of the applications and the method as contemplated herein, what was said above about the cosmetic preparations a) as contemplated herein and the device for flash vaporization b) also applies here, mutatis mutandis.

The agents, applications and methods as contemplated herein and some of their preferred embodiments are characterized in summary form below by the following points:
1. Cosmetic product comprising:
a) a cosmetic preparation containing, based on its total weight
a1) from about 45 to about 99 wt % of at least one polar solvent;
a2) from about 0.1 to about 6.0 wt % cationic polymer;
a3) from about 0.1 to about 12 wt % nonionic polymer;
b) a device for flash vaporization of the cosmetic preparation a).
2. Cosmetic product according to point 1, characterized in that the device for flash vaporization comprises a container b1) and a heating device b2) and is designed so that

the cosmetic preparation a) can be accommodated in the interior of the container b1),

the interior of the container b1), which is at least partially filled with the cosmetic preparation a), can be sealed,

the cosmetic preparation a) can be heated in the closed interior of the container b1) by employing the heating device b2) with an increase in pressure,

the heated cosmetic preparation a) from the interior of container b1) can be released into the surroundings with a reduction in pressure.
3. Cosmetic product according to any one of the preceding points, characterized in that the amount by weight of the polar solvent a1) in the total weight of the cosmetic preparation a) is from about 70 to about 98 wt %, preferably from about 80 to about 95 wt %.
4. Cosmetic product according to any one of the preceding points, characterized in that the polar solvent a1) has a boiling point between about 50 and about 110° C., preferably between about 70 and about 105° C. (20° C., 1013 bar).
5. Cosmetic product according to any one of the preceding points, characterized in that the polar solvent a1) is selected from the group comprised of ethanol, isopropanol and water.
6. Cosmetic product according to any one of the preceding points, characterized in that the amount by weight of water in the total weight of the polar solvent a1) is more than about 80 wt %, preferably more than about 88 wt % and in particular more than about 92 wt %.
7. Cosmetic product according to any one of the preceding points, characterized in that the amount by weight of water and ethanol in the total weight of the polar solvent a1) is at least about 60 wt %, preferably at least about 80 wt %, especially preferably at least about 90 wt % and in particular preferably about 95 wt %.
8. Cosmetic product according to any one of the preceding points, characterized in that the amount by weight of water and ethanol in the total weight of the polar solvent a1) is at least about 60 wt %, preferably at least about 80 wt %, especially preferably at least about 90 wt % and in particular preferably about 95 wt %, wherein the weight ratio of ethanol to water is preferably from about 12:1 to about 4:1, especially from about 10:1 to about 6:1.
9. Cosmetic product according to any one of the preceding points, characterized in that the amount by weight of the cationic polymer a2) in the total weight of the cosmetic preparation a) is from about 0.2 to about 4.0 wt %, preferably from about 0.5 to about 3.0 wt %.
10. Cosmetic product according to point 9, characterized in that the cationic polymer a2) selected from the group of cationic polymers with the INCI designations polyquaternium-4, polyquaternium-11, polyquaternium-16, polyquaternium-55, polyquaternium-69.
11. Cosmetic product according to any one of the preceding points, characterized in that cosmetic preparation a) contains based on its total weight from about 1.0 to about 10 wt %, preferably from about 2.0 to about 8.0 wt % nonionic polymer a3).
12. Cosmetic product according to point 11, characterized in that the nonionic polymer a3) is selected from the group of nonionic polymers, preferably from the group of polyvinylpyrrolidones and vinylpyrrolidone/vinyl acetate copolymers, preferably the vinylpyrrolidone/vinyl acetate copolymers.
13. Cosmetic product according to any one of the preceding points, characterized in that cosmetic preparation a) preferably contains, based on its total weight, from about 0.02 to about 4.0 wt %, preferably from about 0.05 to about 2.0 wt % and especially from about 0.1 to about 1.0 wt % nonionic surfactant a4).
14. Cosmetic product according to point 13, characterized in that the nonionic surfactant is selected from the group of PEG derivatives of hydrogenated castor oil, especially preferably from the group of PEG-40 hydrogenated castor oil and PEG-40 hydrogenated castor oil.
15. Cosmetic product according to any one of the preceding points, characterized in that cosmetic preparation a) preferably contains, based on its total weight, from about 0.05 to about 3.0 wt %, preferably from about 0.1 to about 2.0 wt % and especially from about 0.2 to about 1.0 wt % cationic surfactant a5).
16. Cosmetic product according to point 15, characterized in that the cationic surfactant a5) is selected from the groups of quaternary ammonium compounds, esterquats and amidoamines, preferably from the group of quaternary ammonium compounds, in particular from the group of (C₁₂ to C₁₈) alkyltrimethyl ammonium salt(s).
17. Cosmetic product according to any one of the preceding points, characterized in that the amount by weight of the ingredients a1), a2) and a3) in the total weight of the cosmetic preparation is at least about 80 wt %, preferably at least about 87 wt % and in particular at least about 95 wt %.
18. Use of the cosmetic preparation a) containing, based on its total weight
a1) from about 45 to about 99 wt % of at least one polar solvent;
a2) from about 0.1 to about 6.0 wt % cationic polymer;
a3) from about 0.1 to about 12 wt % nonionic polymer;
as process material in a device for flash vaporization.
19. Use of a product according to any one of points 1 to 17 for treating keratin-containing fibers in particular human hair with a cosmetic preparation a).
20. Use of a product according to any one of points 1 to 17 for temporary shaping of keratin-containing fibers, in particular human hair.
21. Method for temporary shaping of keratin-containing fibers in particular human hair in which the keratin-containing fibers are treated with a cosmetic preparation a) containing, based on its total weight, by employing a device for flash vaporization
a1) from about 45 to about 99 wt % of at least one polar solvent;
a2) from about 0.1 to about 6.0 wt % cationic polymer;
a3) from about 0.1 to about 12 wt % nonionic polymer.
22. Method according to point 21, characterized in that

a partial amount of the cosmetic a) in a storage container is transferred from this storage container in the interior of which a pressure corresponding to ambient pressure prevails, to a container b1);

then the access between the storage container and container b1) is interrupted by a flow control component such that the flow of the cosmetic preparation a) out of the storage container and into the container b1) can be interrupted;

then the cosmetic preparation a) in the container b1) that is sealed with respect to the environment is heated by employing a heating device so that the pressure in the interior of container b1) rises to levels above the ambient pressure, preferably to levels between about 1.1 and about 8 bar, in particular to levels between about 1.2 and about 4 bar;

then the container b1) which is under a pressure above ambient pressure is opened in such a way that it releases at the outlet at least a partial amount, preferably at least about 50 wt %, especially at least about 80 wt % and in particular at least about 90 wt % of the cosmetic preparation in container b1), releasing it out of the container b1) and into the surroundings with a reduction in the prevailing pressure at the point in time of opening of the container.

While at least one exemplary embodiment has been presented in the foregoing detailed description, it should be appreciated that a vast number of variations exists. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. Cosmetic product comprising:
a) a cosmetic preparation comprising, based on its total weight
a1) from about 45 to about 99 wt % of at least one polar solvent,
a2) from about 0.1 to about 6.0 wt % cationic polymer, and
a3) from about 0.1 to about 12 wt % nonionic polymer; and
b) a device for flash vaporization of the cosmetic preparation a).

2. Cosmetic product according to claim 1, wherein the device for flash vaporization comprises a container b1) and a heating device b2) and is designed so that
the cosmetic preparation a) can be accommodated in the interior of the container b1),
the interior of the container b1), which is at least partially filled with the cosmetic preparation a), can be sealed,
the cosmetic preparation a) can be heated in the closed interior of the container b1) by employing the heating device b2) with an increase in pressure, and
the heated cosmetic preparation a) from the interior of container b 1) can be released into the surroundings with a reduction in pressure.

3. Cosmetic product according to claim 1, wherein the amount by weight of the polar solvent a1) in the total weight of the cosmetic preparation a) is from about 70 to about 98 wt %.

4. Cosmetic product according to claim 1, wherein the amount by weight of the cationic polymer a2) in the total weight of the cosmetic preparation a) is about 0.2 to about 4.0 wt %.

5. Cosmetic product according to claim 1, wherein the cationic polymer a2) selected from the group of cationic polymers with the INCI designations polyquaternium-4, polyquaternium-11, polyquaternium-16, polyquaternium-55, polyquaternium-69, and combinations thereof.

6. Cosmetic product according to claim 1, wherein the cosmetic preparation a) comprises, based on its total weight, from about 1.0 to about 10 wt % of nonionic polymer a3).

7. Cosmetic product according to claim 5, wherein the nonionic polymer a3) is selected from the group of nonionic polymers.

8. A cosmetic preparation a) comprising, based on its total weight:
a1) from about 45 to about 99 wt % of at least one polar solvent;
a2) from about 0.1 to about 6.0 wt % cationic polymer; and
a3) from about 0.1 to about 12 wt % nonionic polymer;
wherein the cosmetic preparation a) is utilized as a material in a device for flash vaporization.

9. Cosmetic product according to claim 1, wherein the cosmetic product is utilized for treating keratin-containing fibers with the cosmetic preparation a).

10. A cosmetic product according to claim 1, wherein the cosmetic product is utilized for temporary shaping of keratin-containing fibers.

11. Method for temporary shaping of keratin-containing fibers, the method comprising treating the keratin-containing fibers with a cosmetic preparation a) by employing a device for flash vaporization, the cosmetic preparation a) comprising, based on its total weight:
a1) from about 45 to about 99 wt % of at least one polar solvent;
a2) from about 0.1 to about 6.0 wt % cationic polymer; and
a3) from about 0.1 to about 12 wt % nonionic polymer.

12. Method according to claim 11, wherein:
a partial amount of the cosmetic a) in a storage container is transferred from this storage container in the interior of which a pressure corresponding to ambient pressure prevails, to a container b1);
then the access between the storage container and container b1) is interrupted by a flow control component such that the flow of the cosmetic preparation a) out of the storage container and into the container b1) can be interrupted;
then the cosmetic preparation a) in the container b1) that is sealed with respect to the environment is heated by employing a heating device so that the pressure in the interior of container b1) rises to levels above the ambient pressure;
then the container b1) which is under a pressure above ambient pressure is opened such that it releases at the outlet at least a partial amount of the cosmetic preparation in the container b1), so that the cosmetic preparation comes out of the container b1) and goes into the surroundings with a reduction in the prevailing pressure at the point in time of opening of the container.

13. Cosmetic product according to claim 7, wherein the nonionic polymer a3) is selected from the group of polyvinylpyrrolidones and vinylpyrrolidone/vinyl acetate copolymers, and combinations thereof.

14. Cosmetic product according to claim 13, wherein the nonionic polymer a3) is selected from the group of vinylpyrrolidone/vinyl acetate copolymers and combinations thereof.

15. Cosmetic product according to claim 1, wherein the cosmetic preparation a) comprises, based on its total weight, from about 0.02 to about 4.0 wt % of a nonionic surfactant a4).

16. Cosmetic product according to claim 15, wherein the nonionic surfactant a4) is selected from the group of PEG derivatives of hydrogenated castor oil and combinations thereof.

17. Cosmetic product according to claim 1, wherein the cosmetic preparation a) comprises, based on its total weight, from about 0.05 to about 3.0 wt % of a cationic surfactant a5).

18. Cosmetic product according to claim 17, wherein the cationic surfactant a5) is selected from the group of quaternary ammonium compounds, esterquats, amidoamines, and combinations thereof.

19. Cosmetic product according to claim 18, wherein the cationic surfactant a5) is selected from the group of (C₁₂ to C₁₈) alkyltrimethyl ammonium salt(s) and combinations thereof.

20. Cosmetic product according to claim 1, wherein the amount by weight of the ingredients a1), a2) and a3) in the total weight of the cosmetic preparation is at least about 80 wt %.
